(19) United States
(12) Patent Application Publication
(10) Pub. No.: US 2004/0208798 A1
(43) Pub. Date: Oct. 21, 2004

(54) Adjustable germicidal system

(75) Inventors: Robson L. Splane JR., Granada Hills, CA (US); Richard Fisk, Granada Hills, CA (US); Timothy Jones, San Pedro, CA (US); Forrest B. Fencl, Huntington Beach, CA (US)

Correspondence Address: SOCAL IP LAW GROUP, 310 N. WESTLAKE BLVD. STE 120, WESTLAKE VILLAGE, CA, 91362, US

(73) Assignee: Steril-Aire USA, Inc.

(21) Appl. No.: 10/419,585

(22) Filed: Apr. 21, 2003

(51) Int. Cl. A61L 9/00; B32B 5/02

(52) U.S. Cl. 422/121; 422/24; 422/4; 422/186.3; 250/455.11

(57) ABSTRACT

There is disclosed an adjustable germicidal system to be attached to an air handling apparatus. In one embodiment, an adjustable germicidal system comprises a first socket assembly adjustably coupled to a second socket assembly. The first socket assembly and the second socket assembly may be spatially adjusted with respect to one another in at least one dimension. A germicidal lamp is coupled to each of the socket assemblies. The adjustability of the socket assemblies allows the lamps to be placed in a desired alignment relative to one another and relative to one or more walls of the air handling apparatus. A single power cord is coupled to one of the socket assemblies.

### RELATED APPLICATION INFORMATION

This application is related to application Ser. No. 10/223,433 filed Aug. 19, 2002 entitled “UV Irradiation of Air Handlers and Other Apparatus.”

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by any one of the patent disclosure as it appears in the Patent and Trademark Office patent files or records, but otherwise reserves all copyright and trade dress rights whatsoever.

### BACKGROUND OF THE INVENTION

1. Field Of The Invention

The invention relates to germicidal lamps and germicidal systems that include germicidal lamps.

2. Description Of Related Art

Heating, ventilation and air conditioning (HVAC) units may be augmented with germicidal systems that emit ultraviolet light to eradicate harmful microorganisms in the air, in ducts and on and in HVAC units. Typical germicidal systems comprise a lamp and a fixture. The fixture typically includes an electronic or magnetic ballast. Germicidal systems may have one or more lamps.

The installation of multiple germicidal lamps may be required to obtain effective coverage and treatment. Often, multiple single lamp germicidal systems are installed to achieve sufficient UV light coverage. The use of multiple single lamp germicidal systems requires multiple power outlets. These power outlets may not be readily available, and may inconveniently require the additional expense of installing additional electrical outlets.

In germicidal systems having multiple lamps, the lamps are typically fixed at positions relative to one another in a single plane. In addition, typical germicidal systems having multiple lamps have a single housing with a fully flat surface that abuts the air carrying ducts and other apparatus to which the germicidal system may be attached. To accommodate the varying configurations of air carrying ducts and air treatment units, manufacturers construct multiple models of germicidal systems to be used with the most common air treatment units and the most common air carrying duct arrangements. This causes manufacturers to incur increased design, manufacturing, warehousing and shipping costs.

Information concerning prior art germicidal lamps and systems may be found in U.S. Pat. No. 5,334,347 entitled “Electric Discharge Device,” U.S. Pat. No. 5,866,076 entitled “Single-Ended Germicidal Lamp for HVAC Systems,” and U.S. Pat. No. 6,280,686 entitled “Control of Health Hazards in an Air Handler,” which are co-owned with this application.

### DESCRIPTION OF THE DRAWINGS

The invention will be described by way of exemplary embodiments, but not limitations, illustrated in the accompanying drawings in which like references denote similar elements.

FIG. 1A is a top perspective view of an adjustable germicidal system having sliding socket assemblies.

FIG. 1B is a view of an integrated lamp.

FIG. 2 is a bottom perspective view of the adjustable germicidal system of FIG. 1A having sliding socket assemblies in a first position.

FIG. 3 is a bottom perspective view of the adjustable germicidal system of FIG. 1A having sliding socket assemblies in an extended position.

FIG. 4 is a side view of the adjustable germicidal system of FIG. 1A having sliding socket assemblies in a first position.

FIG. 5 is a side view of the adjustable germicidal system of FIG. 1A having sliding socket assemblies in an extended position.

FIG. 6 is a top perspective view of an adjustable germicidal system having an accordion portion extended in one dimension.

FIG. 7 is a top perspective view of the adjustable germicidal system of FIG. 6 having an accordion portion compacted in one dimension.

FIG. 8 is a top perspective view of a first adjustable germicidal system having a telescoping portion in an extended position.

FIG. 9 is a top perspective view of the adjustable germicidal system of FIG. 8 having a telescoping portion in a compacted position.

FIG. 10 is a top perspective view of a second adjustable germicidal system having a telescoping portion in a compacted position.

FIG. 11 is a top perspective view of the adjustable germicidal system of FIG. 10 having a telescoping portion in a partially extended position.

FIG. 12 is a top perspective view of the adjustable germicidal system of FIG. 10 having a telescoping portion in a fully extended position.

FIG. 13 is a top perspective view of an adjustable germicidal system having interlocking portions in an extended position.

FIG. 14 is a top perspective view of the adjustable germicidal system of FIG. 13 having interlocking portions in a compacted position.

FIG. 15 is a top perspective view of an adjustable germicidal system having hinged panels shown attached to a flat duct.

FIG. 16 is a top perspective view of the adjustable germicidal system of FIG. 15 having hinged panels shown attached to a corner duct.

FIG. 17 is a top perspective view of an adjustable germicidal system having an extensible coiled cord shown attached to a flat duct.

FIG. 18 is a top perspective view of the adjustable germicidal system of FIG. 17 having an extensible coiled cord shown attached to the side of a graded duct.

FIG. 19 is a top perspective view of the adjustable germicidal system of FIG. 17 having an extensible coiled cord shown attached to panels of a graded duct.

FIG. 20 is a top perspective view of an adjustable germicidal system having a cross arm attached with hinges to socket assemblies.

FIG. 21 is a top perspective view of an adjustable germicidal system having a circular cross panel attached with hinges to elongated socket assemblies.

FIG. 22 is a top perspective view of an adjustable germicidal system having an accordion portion shown in an extended position attached to a wall of an air handling apparatus.

FIG. 23 is a top perspective view of an adjustable germicidal system having a flexible tube connecting the socket assemblies.

### DETAILED DESCRIPTION OF THE INVENTION

Throughout this description, the embodiments and examples shown should be considered as exemplars, rather than limitations on the apparatus and methods of the invention.

The various embodiments of the invention described herein allow for the installation of a spatially adjustable germicidal system to air carrying ducts and to air treating units, such as heating, ventilation and air conditioning (HVAC) units as well as humidifier and dehumidifier systems and other air handling systems, devices, and related ducts. As used herein, the term air handling apparatus refers to and includes HVAC units of all kinds such as, but not limited to, air conditioners, heaters, humidifiers, and dehumidifiers, installed as a single unit, as multiple units and in combination, as well as any air treatment or air delivery device and associated ducts.

At many sites in which air handling apparatus are installed, the surface configuration of the walls of the air handling apparatus may not allow for efficient installation of off the shelf prior art germicidal systems. As is set forth in the prior art cited above, there is a preferred spacing and alignment of germicidal lamps in relation to the air handling apparatus when applying ultraviolet (UV) radiation to the air located in and/or on the surface of the air handling apparatus. The invention described herein provides various embodiments of spatially adjustable germicidal systems which may be installed with air handling apparatus in a multitude of configurations and alignments to achieve an effective amount of coverage of UV radiation from the germicidal lamps included in the germicidal system.

To overcome the limitations of the prior art and to better meet the diverse requirements of air handling apparatus provided by a multitude of manufacturers, the invention described herein provides various embodiments of an adjustable germicidal system. The adjustable germicidal systems described herein may be coupled to the walls of a variety of air treatment units, to a plethora of air carrying duct configurations, and to other air handling apparatus. The adjustable features of the germicidal systems described herein eliminate the need to manufacture a variety of fixed germicidal systems required to be used with differing air treatment units and differing duct configurations.

The adjustable germicidal systems may be installed on non-parallel planes, and adapted to change in one, two or three dimensions. Furthermore, the adjustable germicidal system may be adjusted in one or more axes, and is some embodiments, infinite axes. The adjustable features of the germicidal systems described herein allow for customization and adjustment of the germicidal systems at the site of air handling apparatus by installers and other users immediately prior to installing the germicidal systems. Further, each of the adjustable germicidal systems described herein may be adjusted without the use of tools.

In addition, the features of some of the embodiments of the germicidal systems described herein allow for attachment to walls of air handling apparatus that are not entirely flat, and may be curved or have stepped or angled walls.

The features of each of the embodiments of the adjustable germicidal systems described herein may be mixed with and incorporated in combination with the features of the other embodiments of the adjustable germicidal systems described herein.

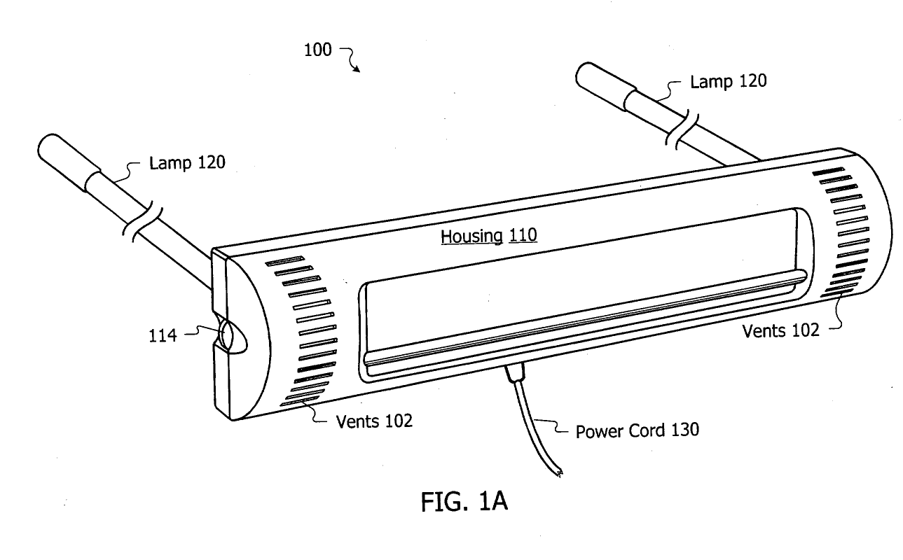

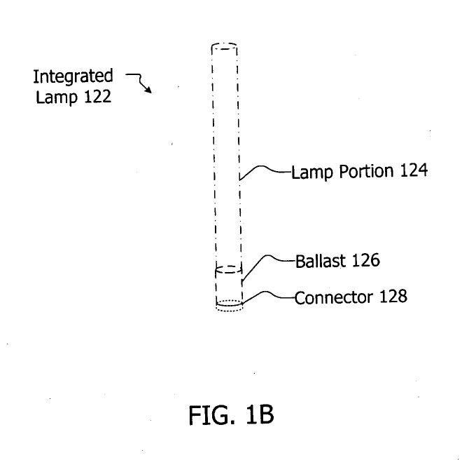

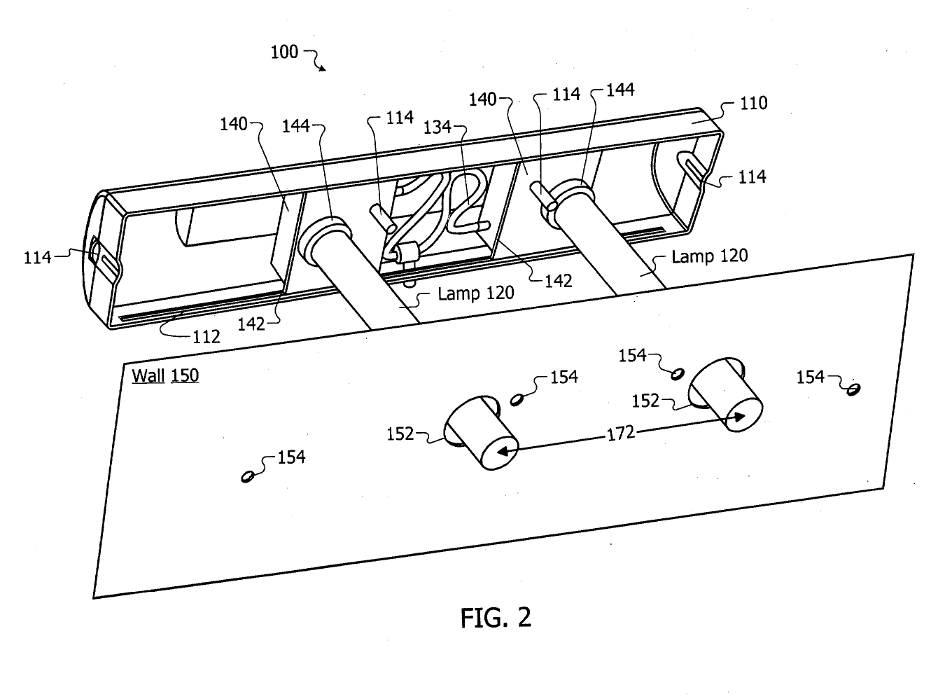

FIG. 1A is a top perspective view of adjustable germicidal system 100, and FIGS. 2 and 3 are bottom perspective views of the adjustable germicidal system 100 shown in FIG. 1A.

The germicidal system may include two or more germicidal UV lamps. Each of the germicidal UV lamps 120 is coupled to a socket assembly 140.

The germicidal UV lamps 120 may be those UV lamps that emit sufficient UV-C radiation to eradicate airborne germs and germs that accumulate in the air in and on the internal surfaces of air handling apparatus. In one embodiment, the UV lamps 120 emit broadband UV-C in the range of 250-260 nm. In one embodiment, the UV lamps 120 emit UV-C radiation substantially at 253.7 nm. The UV lamps 120 generate an insignificant quantity or less of ozone. The UV lamps 120 are single ended. In one embodiment, the UV lamps are the single ended tubes described in U.S. Pat. No. 5,866,076 entitled “Single-Ended Germicidal Lamp for HVAC Systems.” Single tube and multiple tube single ended germicidal UV lamps as well as u-shaped, j-shaped and other shaped single ended germicidal UV lamps may be incorporated in the germicidal systems described herein. The UV lamps may be germicidal lamps sold by the assignee of this patent application, Steril-Aire U.S.A., Inc. Other single ended germicidal lamps may be used. The germicidal lamps may have a T5, T8, or other diameter. The germicidal lamps may have a medium bi-pin base, a base that conforms to an industry standard base such as the circline base, or any other base or connector that allows for coupling to the socket assemblies 140. The germicidal lamps 120 may have any appropriate length, such as, for example, without limitation, 12″, 16″, 20″, 24″, 30″, 36″, 40″, 48″, 56″ and 64″.

FIG. 1B is a view of an integrated lamp 122. The integrated lamp 122 includes a lamp portion 124 and a ballast device 126 integrated therewith. In this way, the germicidal UV lamp is an integrated germicidal UV lamp. The end of the integrated germicidal UV lamp that includes ballast device 126 is referred to herein as the ballast end of the integrated lamp. Connector 128 is coupled to the ballast device 126 of the integrated lamp 122. In one embodiment, connector 128 is used to couple the integrated lamp 122 to the socket assemblies 140. In one embodiment, lamp 120 may be an integrated lamp 122.

Although not depicted with each of the embodiments described herein, each of the lamps included in the various embodiments of the germicidal systems may be an integrated germicidal UV lamp or a non-integrated germicidal UV lamp. As used herein, the term lamp refers to single ended germicidal UV lamps as described in the prior two paragraphs. The term lamp encompasses both germicidal UV lamps and integrated germicidal UV lamps.

The socket assemblies 140 are included in the housing 110. In one embodiment, two germicidal lamps 120 are coupled to two socket assemblies 140 included in housing 110. The socket assemblies 140 each include a receiving socket 144 or other receiving means to which the germicidal lamps 120 are coupled.

In one embodiment, the receiving socket 144 is a coordinated receiving socket that corresponds to a matching coordinated connector that is included with the lamp 120. The receiving socket 144 may include a ceramic or other nonconductive sleeve having a metal or other conductive portion. The metal or other conductive portion is constructed to correspond to the connector of lamp 120 for the passing of electrical current from the socket assemblies 140 to the lamps 120.

In one embodiment, the socket assemblies 140 have included therein a ballast device (not shown). The ballast device may be any electrical germicidal lamp energizing device. In this embodiment, the lamps 120 are not integrated germicidal UV lamps.

In another embodiment, the socket assemblies 140 do not have a ballast device included therein. In this embodiment, the lamps 120 are integrated germicidal UV lamps 122. In this embodiment, receiving socket 144 of the socket assembly 140 is coupled to the ballast end of the integrated lamp 122.

Each of the housings, including housing 110, socket assemblies and socket assembly covers described herein including arms, interlocking portions, telescoping portions and various other components of the various embodiments of germicidal systems described hereinbelow may be manufactured from any resilient, strong, durable material that remains functional despite temperature changes within a normal operating temperature range, such as, for example, but not limited to, metals, plastics, and resins, unless otherwise specified.

Housing 110 may include vents 102 which allow for heat to escape, such as that generated by the socket assemblies 140 and any included ballast device.

In germicidal system 100, the socket assemblies 140 include edge 142 which sits in tracks 112 of housing 110. Edge 142 sits in track 112 to movably hold socket assemblies 140 inside housing 110. Edge 142 may be considered a slot, and track 112 may be considered a groove, such that the configuration may be considered a slot and groove arrangement. The socket assemblies 140 slide along tracks 112 on edges 142 to move closer to and farther from one another. As such, the movement is along a single axis between the socket assemblies 140. Track 112 is located at the outer edge of each of the two longer sides of the housing 110. (Note that only one track 112 is visible in FIGS. 2 and 3). The spacing between the socket assemblies 140 is adjustable to accommodate varying distances between UV lamps 120. This allows the adjustable germicidal system 100 to be installed on the walls of various configurations of air handling apparatus. In one embodiment, no tools are required to move, slide or otherwise adjust the position of the socket assemblies.

As shown in FIGS. 2 and 3, the socket assemblies 140 may be aligned in a variety of positions to allow for a desired spacing of the lamps 120. The socket assemblies 140 and lamps 120 may be positioned a desired distance apart, as limited by the length of the interior of housing 110. The lamps 120 are placed in receiving holes 152/162 in the wall 150 of an air handling apparatus at a position to achieve the desired germicidal effect on the air within and/or internal surfaces of the duct or unit. An installer of the adjustable germicidal system 100 may drill or otherwise create the receiving holes 152/162 in the wall 150 of the air handling apparatus. In each of the other embodiments of the germicidal systems described herein, an installer may drill or otherwise create the receiving holes in the one or more walls of the air handling apparatus to which the particular adjustable germicidal system is to be installed.

The receiving sockets 144 may be flush to the socket assembly 140. In this embodiment, when the germicidal system is installed on wall 150, the receiving sockets 144 are situated on an outside side of a wall 150 of an air handling apparatus. The lamps 120 pass through a receiving hole 152/162 from an inside side of the wall 150 through the wall 150 to be coupled to the receiving socket 144 of socket assembly 140 on the outside side of the wall 150. In this embodiment, the receiving holes 152 and 162 have a diameter sufficient to accommodate the lamps 120. In one embodiment, the receiving holes 152 and 162 are approximately one inch in diameter.

In one embodiment, the receiving sockets 144 extend from the socket assembly 140. In this embodiment, when the germicidal system 100 is installed on wall 150, the receiving sockets 144 are situated on an outside side of a wall 150 of an air handling apparatus and pass through the receiving hole 152/162 to an inside side of the wall 150. In this embodiment, the receiving holes 152 and 162 should be of a diameter sufficient to accommodate the receiving sockets 144. The lamps 120 are coupled to the receiving sockets 144 of socket assembly 140. The lamps 120 are on the inside side of the wall 150. More specifically, the connector of the lamps 120 are on either the inside side or the outside side of the wall 150, depending on the depth of the receiving socket 144 and the amount the receiving socket 144 extends through the wall 150 into the inside side of the wall 150.

In each of the embodiments of the germicidal systems described hereinbelow the receiving sockets may be flush to the socket assemblies and the receiving sockets may extend from the socket assemblies as described in the prior two paragraphs.

FIG. 2 shows the lamps 120 and corresponding socket assemblies 140 relatively close together, such that the lamps 120 and the receiving holes 152 are a distance 172 apart. FIG. 3, shows the lamps 120 and the socket assemblies 140 further apart, such that the lamps 120 and the receiving holes 162 are a distance 174 apart.

In one embodiment, housing 110 of adjustable germicidal system 100 is attached to an air handling apparatus wall 150 via an attachment receptacle 114 in the housing 110 and corresponding receiving holes 154 (FIG. 2) or 164 (FIG. 3). The attachment may be made using screws, bolts, rivets, and other fasteners. As shown in FIGS. 2 and 3, the housing 110 may include receptacles 114 through which screws or other fastening devices may be passed so that the germicidal system may be attached to duct wall or air treatment unit wall 150. Wall 150 may have corresponding holes 154 and 164 or other fastener receivers by which housing 110 is attached to the duct 150. In other embodiments, the housing 110 may be attached to wall 150 using adhesives, clips, hook and loop fasteners such as Velcro® brand hook and loop fasteners. Each of the embodiments of the germicidal systems described hereinbelow may be attached as described in this paragraph, and in other ways.

As described herein, each of the embodiments of the germicidal systems receive electrical power via a single power cord coupled to one of the socket assemblies, such as, for example, power cord 130. In each of the embodiments of the germicidal systems described herein, power may be passed from one socket assembly to the next socket assembly . For example, the power is passed from one socket assembly 140 to the other socket assembly 140 via internal power cord 134. In various embodiments, this is achieved via an internal power cord, such as internal power cord 134, or a power channel, power line or other power carrying medium included in the portion of the germicidal system that couples the socket assemblies to one another. In this way, each of the socket assemblies receive power from a single source, although the power cord is only coupled to one of the socket assemblies of the germicidal system. Each of the embodiments of the germicidal systems described herein incorporate a power passing means as described in this paragraph.

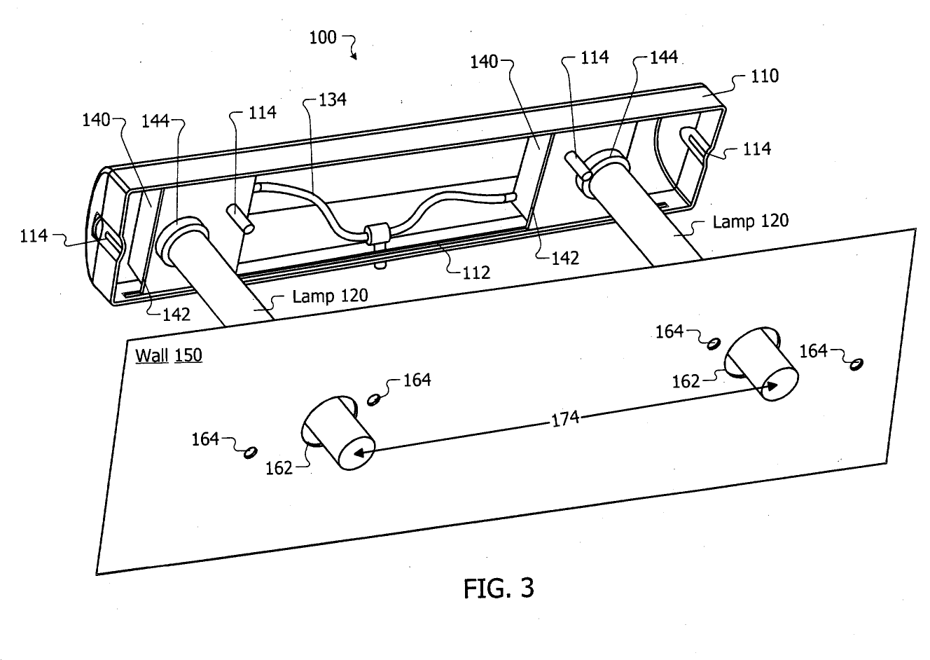

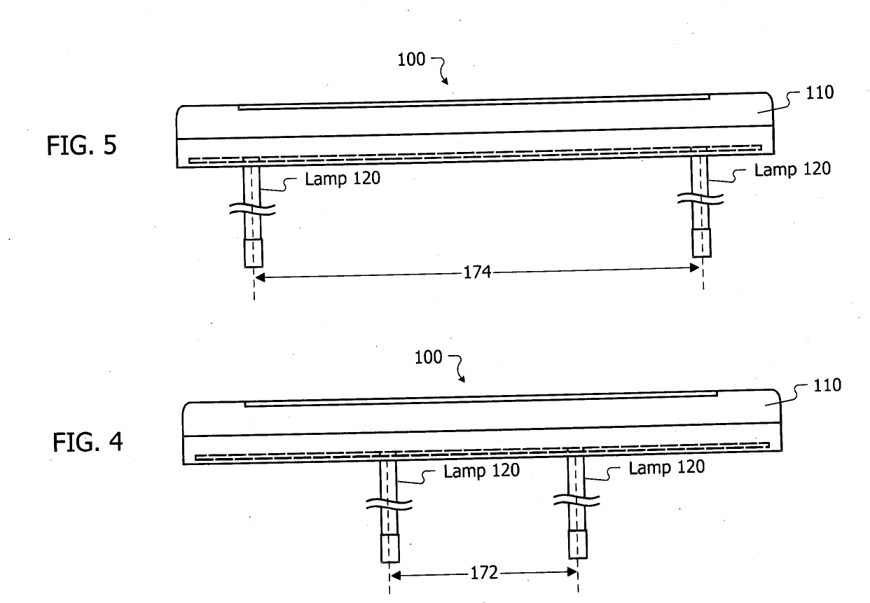

FIG. 4 is a side view of adjustable germicidal system 100 in a compact or close position such that the lamps 120 are a distance 172 apart. FIG. 5 is a side view of adjustable germicidal system 100 in an extended position such that the lamps 120 are a distance 174 apart. FIGS. 4 and 5 correspond to side views of the adjustable germicidal system 100 shown in FIGS. 2 and 3.

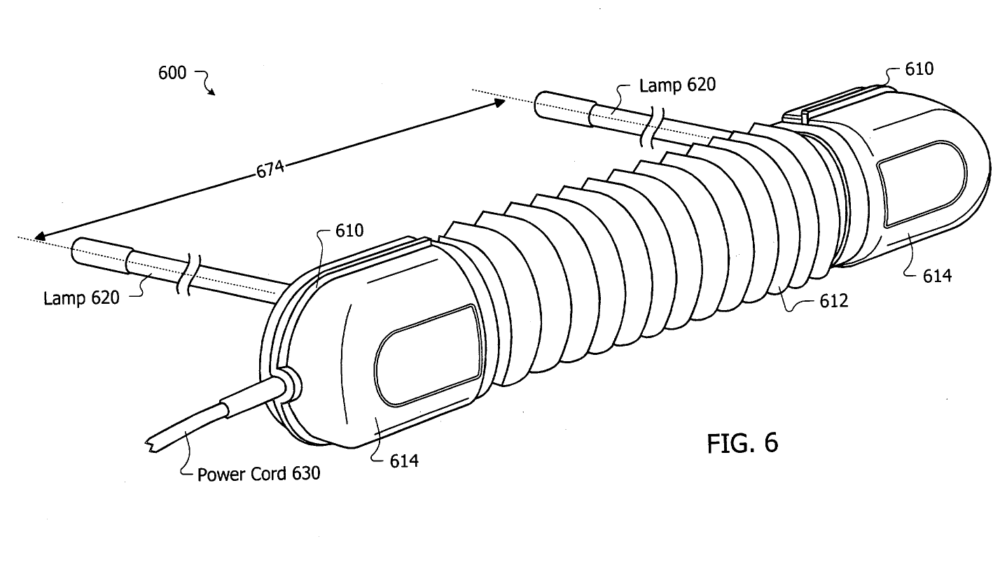

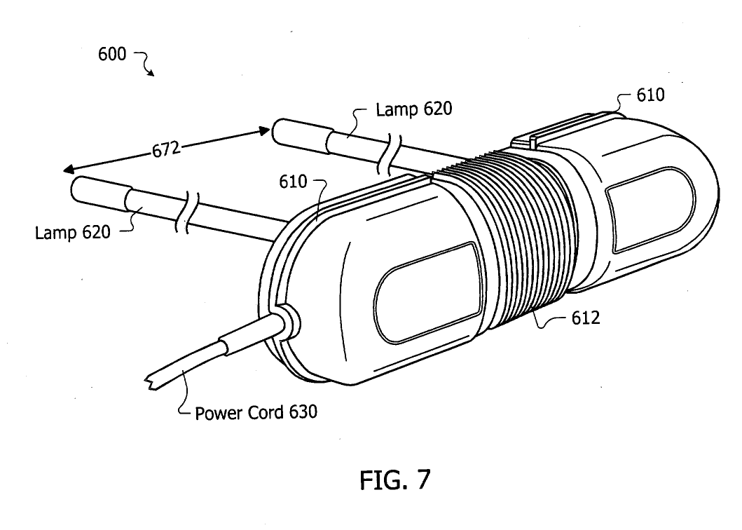

FIG. 6 is a top perspective view of an adjustable germicidal system 600 having an extendable accordion portion 612 in an extended position. FIG. 7 is a top perspective view of adjustable germicidal system 600 having an extendable accordion portion 612 in a compacted position. In this embodiment, socket assemblies 610 are coupled to one another via an extendable accordion portion 612 which allows the lamps 620 to be adjusted in three dimensions in relation to one another. In this way, the lamps 620 may be positioned a desired distance apart and the accordion portion 612 may be twisted or bent. In some applications it may be desirable to position the lamps 620 a greater distance 674 apart, as shown in FIG. 6. In other applications, it may be desirable to set the lamps 620 a shorter distance 672 apart, as shown in FIG. 7. In one embodiment, no tools are required to move, slide or otherwise adjust the position of the socket assemblies 610.

The lamps 620 may receive electrical power via power cord 630 which is coupled to one of the socket assemblies 610.

The accordion portion 612 may be manufactured from any resilient, strong, durable material that is flexible and remain functional despite temperature changes within a normal operating temperature range, such as, for example, but not limited to, plastics, resins, and metal meshes.

In this embodiment, the socket assemblies may include a cover 614. Either the entire cover 614 or a portion of the cover of socket assembly 610 may be removed to provide access to the lamp 620. In this way, the lamp 620 may be replaced while the germicidal system is attached to an air handling apparatus wall by opening the cover 614 to access the lamp 620. In all embodiments of the germicidal systems discussed herein below, either the entire cover or a portion of the cover of the various socket assemblies may be removed to provide access to the lamp included therein. In this way, the lamps may be replaced while the germicidal system is attached to an air handling apparatus wall. That is, the cover of the socket assemblies may be opened or removed, the lamp may be removed, and a replacement lamp may be installed.

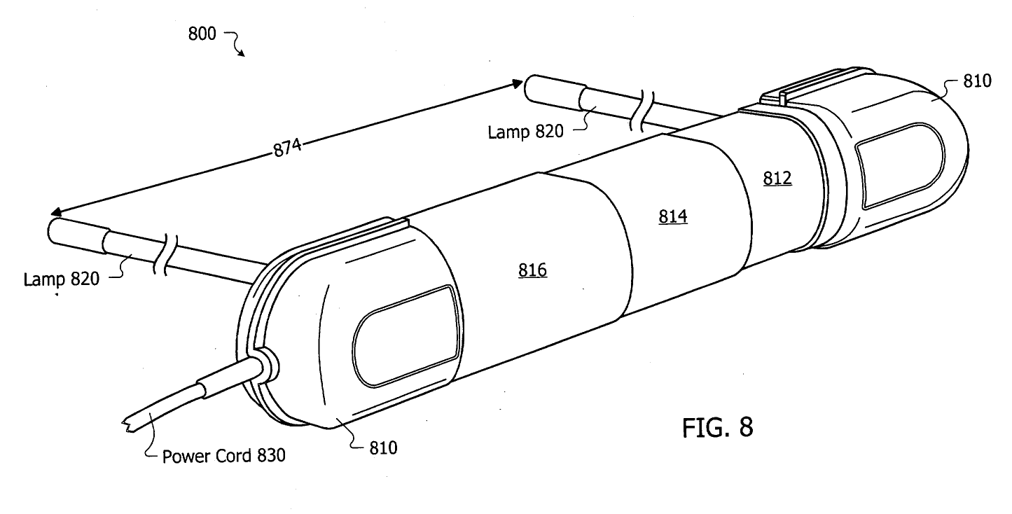

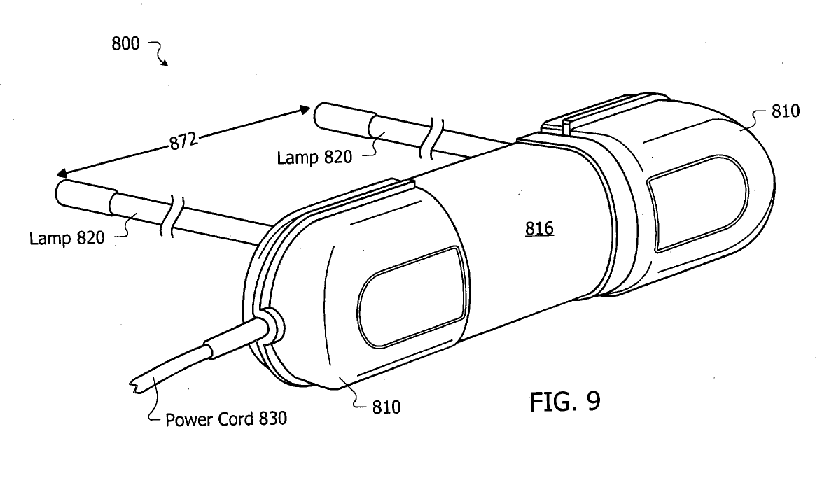

FIG. 8 is a top perspective view of a first adjustable germicidal system 800 having telescoping portions 812, 814 and 816 in an extended position. FIG. 9 is a top perspective view of adjustable germicidal system 800 having telescoping portions in a compacted position. In this embodiment, socket assemblies 810 are coupled to one another via telescoping portions 812, 814 and 816.

Lamps 820 may be adjusted in one dimension, along a single axis, in relation to one another. In this way, the lamps 820 may be set a desired distance apart by moving the socket assemblies 810 closer together or farther apart. In some applications it may be desirable to set the lamps 820 a greater distance 874 apart, as shown in FIG. 8. In other applications, it may be desirable to set the lamps 820 a shorter distance 872 apart, as shown in FIG. 7. When arranged in its most compact position so that the lamps 820 are shorter distance 872 from one another, the telescoping portions 812 and 814 are, in one embodiment, underneath or behind topmost telescoping portion 816, such that telescoping portions 812 and 814 cannot be seen.

The lamps 820 may receive electrical power via power cord 830 which is coupled to one of the socket assemblies 810.

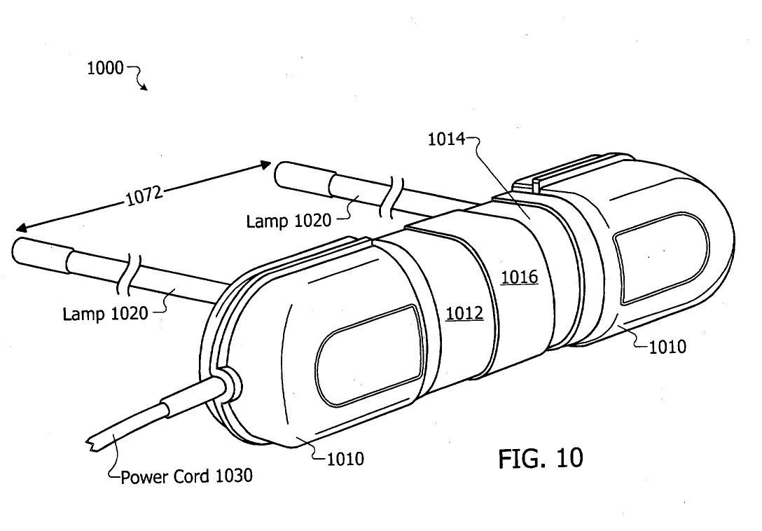

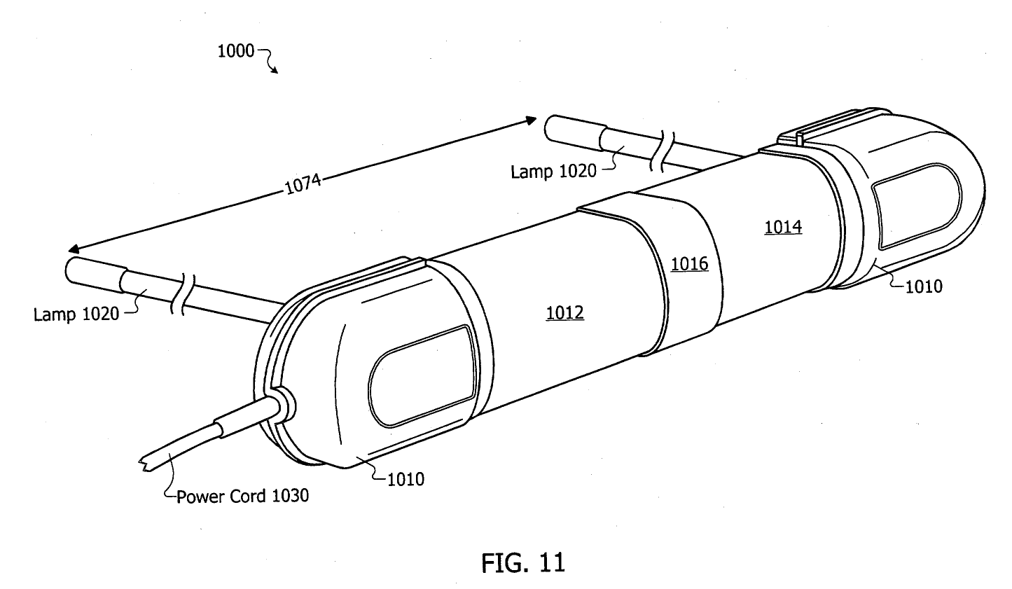

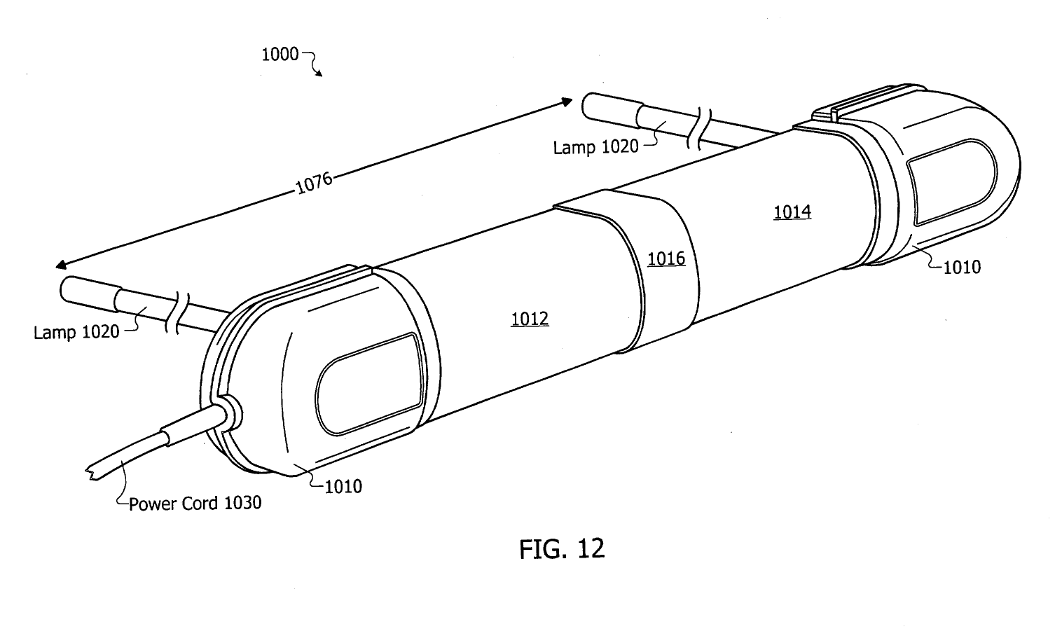

FIG. 10 is a top perspective view of a second adjustable germicidal system 1000 having telescoping portions in a compacted position. FIG. 11 is a top perspective view of adjustable germicidal system 1000 having telescoping portions in a partially extended position. FIG. 12 is a top perspective view of adjustable germicidal system 1000 having telescoping portions in a fully extended position. In this embodiment, socket assemblies 1010 are coupled to one another via telescoping portions 1012 and 1014. Both telescoping portions 1012 and 1014 may slide under stationary support 1016. In one embodiment, stationary support 1016 holds the telescoping portions 1012 and 1014 flush to the system 1000. In one embodiment, telescoping portions 1012 and 1014 slide in and out of an opening in socket assemblies 1010 to allow the lamps 1020 to be moved closer together and farther apart. In another embodiment, the telescoping portions 1012 and 1014 are each fixed to a socket assembly 1010. The telescoping portion 1014 may slide under telescoping portion 1012 to allow for adjustment of the distance between lamps 1020 and the socket assemblies 1010.

Lamps 1020 may be adjusted in one dimension in relation to one another. To position the lamps 1020 a desired distance apart, the socket assemblies 1010 are moved closer together and farther apart without the need of tools. The socket assemblies 1010 and telescoping portions 1012 and 1014 may include positioning apparatus such as extensions and détentes at spaced, pre-defined positions to allow the distance 1072 to be adjusted with precision. The positioning apparatus may also take other forms. In some applications it may be desirable to set the lamps 1020 a greater distance 1074 apart, as shown in FIG. 11, or to a maximum extended position to allow for the greatest distance 1076 apart. In other applications, it may be desirable to set the lamps 1020 a shorter distance 1072 apart, as shown in FIG. 10. When arranged in its most compact position so that the lamps 1020 are shorter distance 1072 from one another, the telescoping portion 1014 is, in one embodiment, underneath or behind the telescoping portion 1012.

The lamps 1020 may receive electrical power via power cord 1030 which is coupled to one of the socket assemblies 1010.

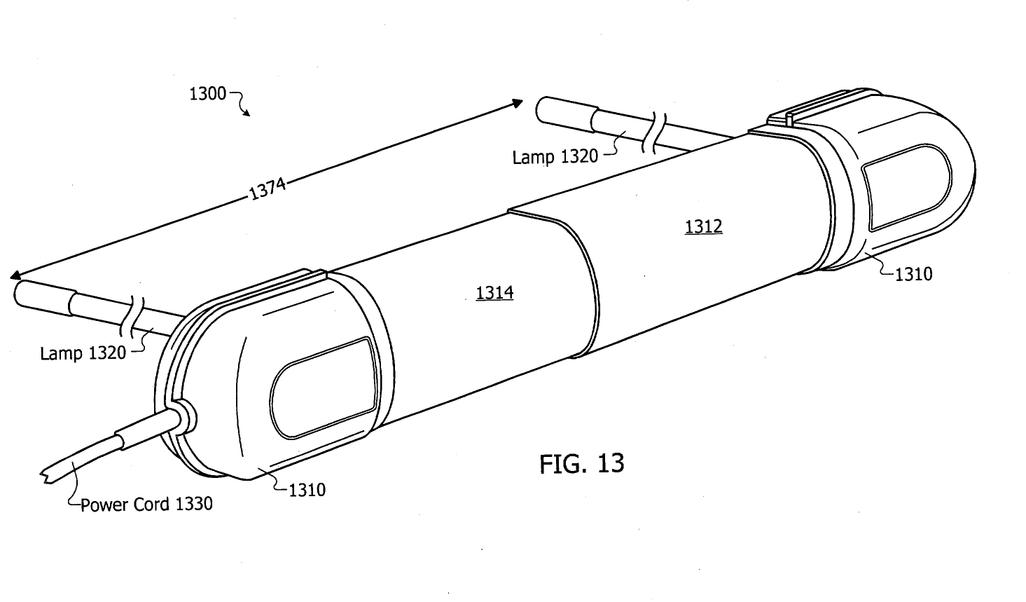

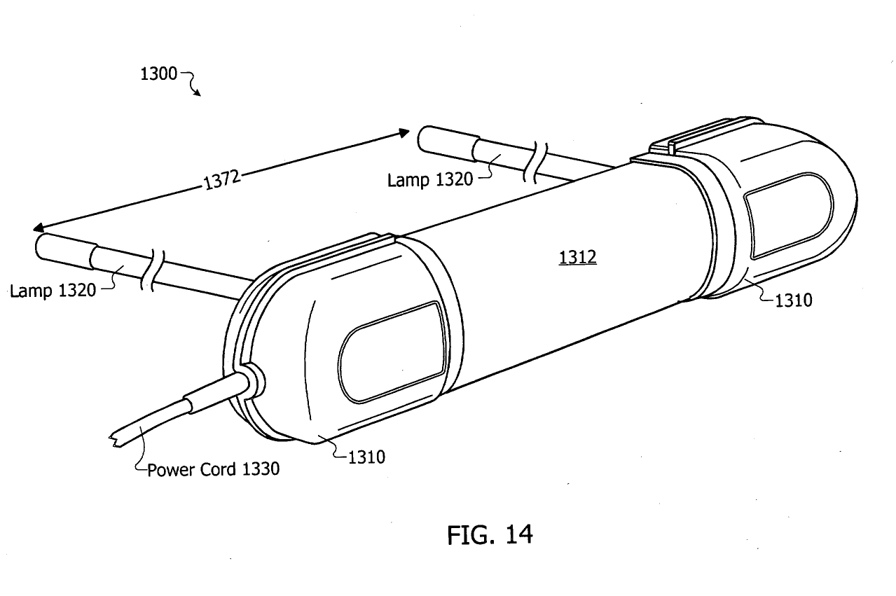

FIG. 13 is a top perspective view of an adjustable germicidal system 1300 having interlocking portions 1312 and 1314 in an extended position. FIG. 14 is a top perspective view of adjustable germicidal system 1300 having interlocking portions 1312 and 1314 in a compacted position. In this embodiment, socket assemblies 1310 are coupled to one another via interlocking portions 1312 and 1314. Interlocking portions 1312 and 1314 may be coupled to socket assemblies 1310. Interlocking portion 1314 slides under interlocking portion 1012 when the socket assemblies 1310 are moved closer together. In one embodiment, the longer edges, the longitudinal edges, of interlocking portions 1312 and 1314 overlap with a bottom (not shown) of the adjustable germicidal system 1300. In this way, the interlocking portions 1312 and 1314 are movably secured to the base or bottom of the germicidal system 1300.

Lamps 1320 may be adjusted in one dimension in relation to one another. The distance between lamps 1320 may be adjusted. The lamps 1320 may be set a desired distance apart by sliding the socket assemblies 1310 closer together and farther apart. In some applications it may be desirable to set the lamps 1320 a greater distance 1374 apart, as shown in FIG. 13. In other applications, it may be desirable to set the lamps 1320 a shorter distance 1372 apart, as shown in FIG. 14. When arranged in its most compact position so that the lamps 1320 are shorter distance 1372 from one another, the interlocking portion 1314 is, in one embodiment, underneath or behind-the interlocking portion 1312. In various applications, the lamps 1320 may be adjusted to a desired distance from one another between greater distance 1374 and shorter distance 1372. The length of the interlocking portions 1312, 1314 may be decreased in the field by cutting to the desired length. One or both of the interlocking portions 1312, 1314 may be perforated or scored so that their length may be shortened in the field without tools. The length of the accordion portion 612 of FIG. 6 may also be shortened in the same manner.

The lamps 1320 may receive electrical power via power cord 1330 which is coupled to one of the socket assemblies 1310.

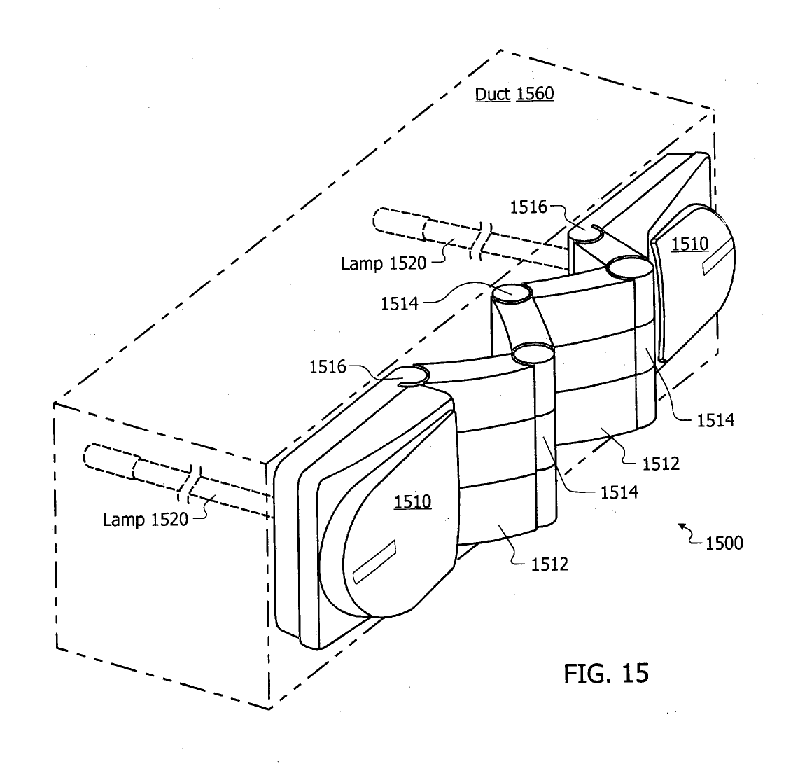

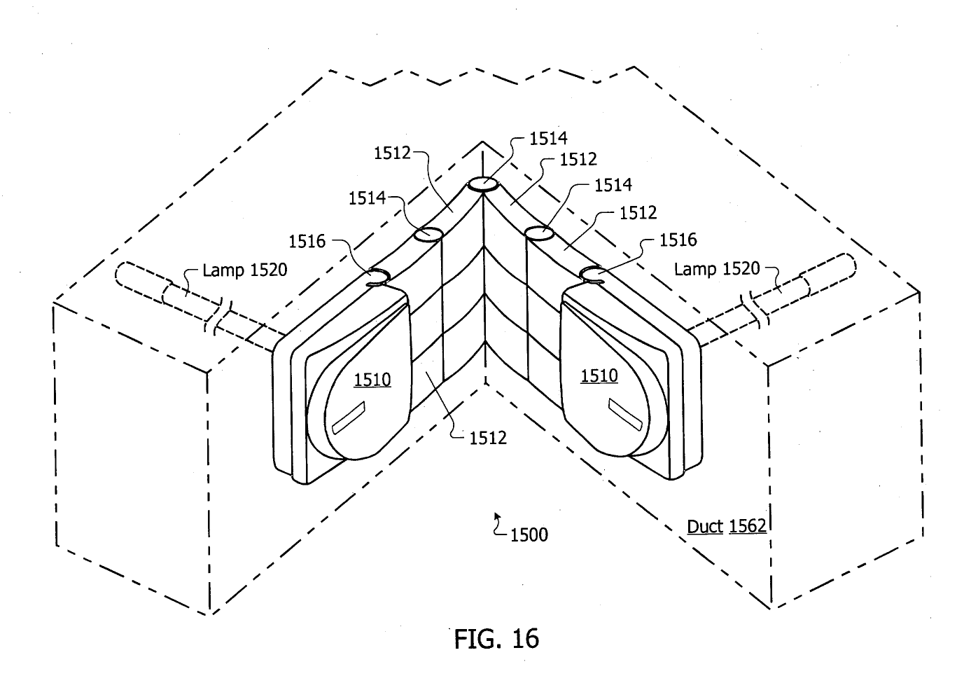

FIG. 15 is a top perspective view of an adjustable germicidal system 1500 having hinged panels 1512 shown attached to a flat duct 1560. FIG. 16 is a top perspective view of adjustable germicidal system 1500 having hinged panels 1512 shown attached to a comer duct 1562. In one embodiment, germicidal system 1500 includes three hinged panels 1512 coupled between two socket assemblies 1510. Each of the socket assemblies 1510 has a lamp 1520 attached thereto. In other embodiments, two or more hinged panels 1512 may be included, such as for example, two, four, five and six hinged panels 1512. In one embodiment, the hinged panels 1512 are equal in size, and, in another embodiment, the hinged panels may have differing lengths when compared to each other.

The hinged panels 1512 may be coupled to socket assemblies 1510 via hinge portion 1516 of socket assembly 1510. The hinged panels 1512 may be coupled to neighboring hinged panels 1512 via hinges 1514. The hinged panels 1512 may be folded at hinges 1514 to move the socket assemblies 1510 closer together and farther apart. This allows for adjustment of the distance between lamps 1520. When the panels 1523 are flush to the surface of a duct, the lamps 1520 are the maximum distance apart. FIG. 15 depicts the germicidal system 1500 in a partially extended position. The adjustable nature of the germicidal system 1500 allows for installation at various sites having varying configured air handling apparatus.

In germicidal system 1500, lamps 1520 may be adjusted in two dimensions in relation to one another. This may be seen in FIG. 16 in which germicidal system 1500 is folded or bent to conform to the square or angled corner of duct 1562. In this example, the lamps 1520 may be set at desired locations by bending the germicidal system 1500 at hinges 1514 between hinged panels 1512. The adjustable germicidal system 1500 may be installed in a variety of surface locations, including, but not limited air handling apparatus having flat adjacent walls that are at an angle from one another. For example, the walls of duct 1562 are approximately 90 degrees apart. In other example configurations the walls of an air handling apparatus may be at an angle of 30, 45, 60, etc. degrees from one another.

In addition, adjustable germicidal system 1500 may be installed in air handling apparatus walls that have flat portions separated by a curved portion. In this kind of installation, the flat wall portions are needed for the flush installation of the socket assemblies 1510, even though the area between the flat wall portions may be curved, angled, or in a varied configuration.

In a related embodiment, the socket assemblies 1510 may have a generally pliable resilient rubber, foam or other back mounting surface which allows for attachment to curved, ridged, ribbed, bumpy, rough and other more difficult to mount air handling apparatus.

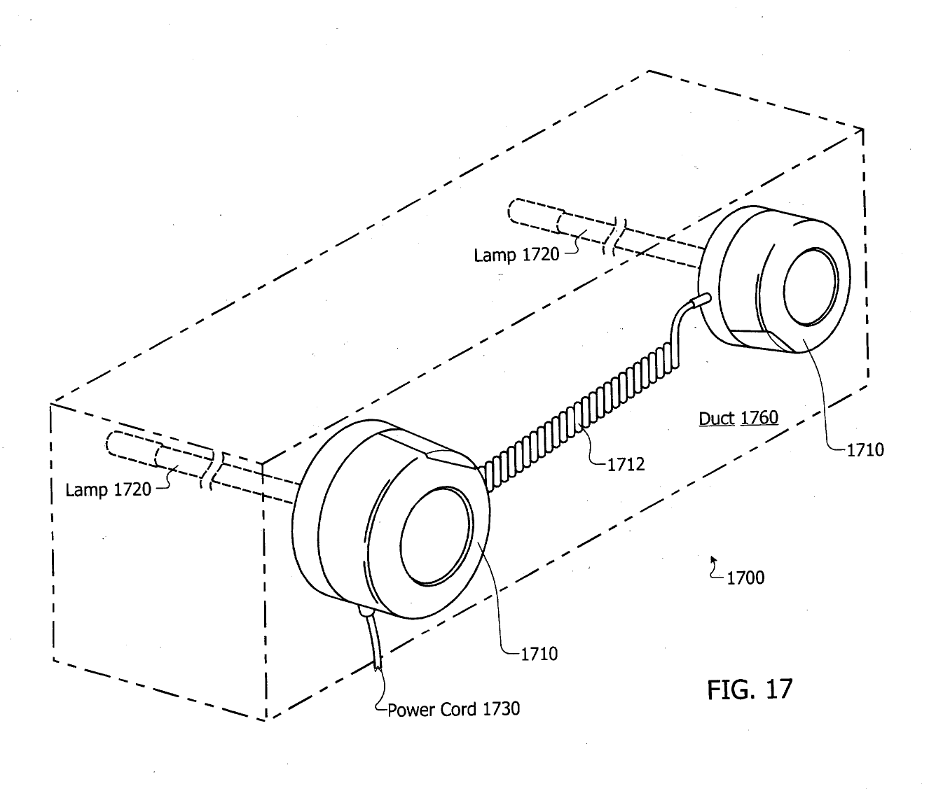

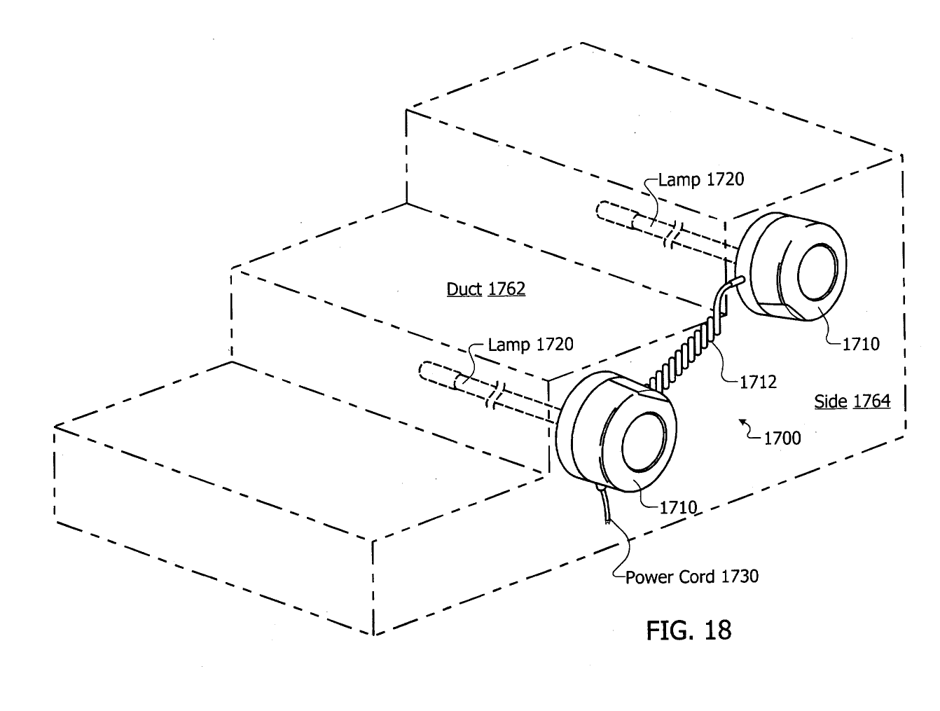

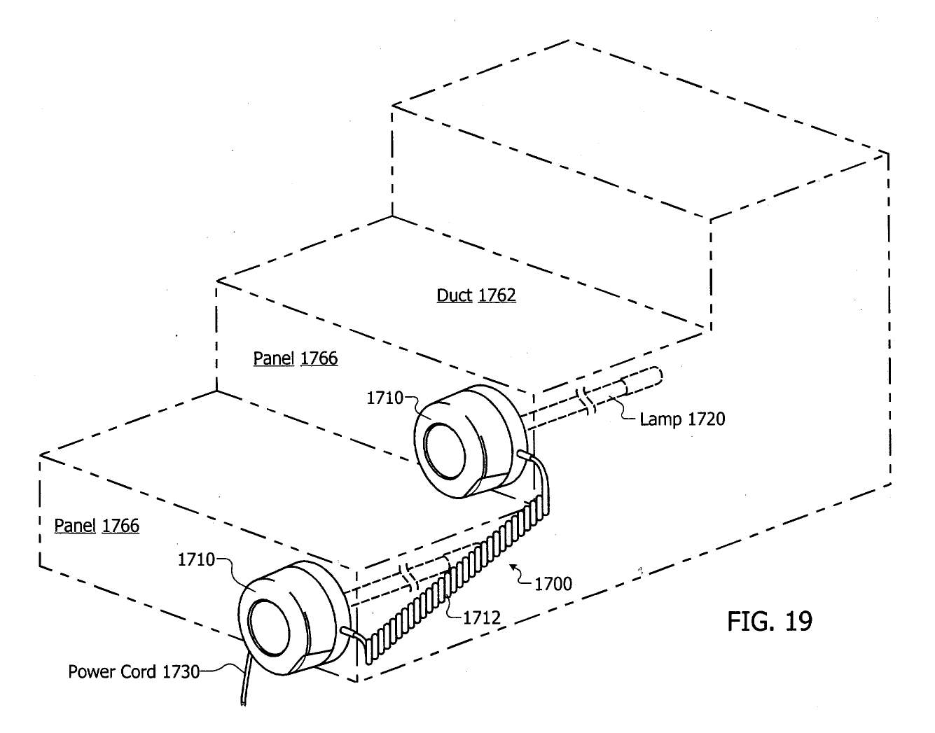

FIG. 17 is a top perspective view of an adjustable germicidal system 1700 having an extensible coiled cord 1712 shown attached to a flat duct 1760. FIG. 18 is a top perspective view of adjustable germicidal system 1700 having an extensible coiled cord 1712 shown attached to the side 1764 of a graded duct 1762. FIG. 19 is a top perspective view of adjustable germicidal system 1700 having an extensible coiled cord 1712 shown attached to panels 1766 of the graded duct 1762. In one embodiment, adjustable germicidal system 1700 includes two socket assemblies 1710 coupled to one another via an extensible coiled cord 1712. In other embodiments, more than two socket assemblies 1710 may be included in adjustable germicidal system 1700 such that each socket assembly 1710 is coupled to its neighbor by a length of extensible coiled cord 1712. Each of the socket assemblies 1710 have a lamp 1720 attached thereto.

The extensible coiled cord 1712 allows for the distance between lamps 1720 to be adjusted to meet a variety of installation configurations. The extensible coiled cord 1712 may be a cord that behaves in a manner similar to an extensible telephone cord that attaches a handset to a telephone base unit or cradle. The extensible coiled cord 1712 allows the relation of the lamps 1720 to one another and to the interior of the duct to be arranged by allowing the socket assemblies 1710 to be moved closer together and farther apart. As such, no tools are required to move, slide or otherwise adjust the position of the socket assemblies 1710.

The extensible coiled cord 1712 also allows the socket assemblies 1710 to be attached to air handling apparatus walls that are in different planes. The extensible coiled cord 1712 allow the lamps 1720 to be adjusted in three dimensions, in infinite axes, in relation to one another. The adjustable nature of the germicidal system 1700 allows for installation to a variety of different air handling apparatus having differing lamp placement requirements and installation limitations.

By virtue of the extensible coiled cord 1712 by which the socket assemblies 1710 are attached, the adjustable germicidal system 1700 may be installed in a variety of surface locations, including, but not limited to air handling apparatus walls that are flat, angled and/or curved. In one embodiment, generally flat wall portions are needed for the flush installation of the socket assemblies 1710, even though the area between the flat wall portions may be curved, angled, or in a varied configuration. In a related embodiment, the socket assemblies 1710 may have a generally pliable resilient rubber, foam or other back mounting surface which allows for attachment to curved, ridged, ribbed, bumpy, rough and other more difficult to mount air handling apparatus.

In one embodiment, three or more socket assemblies 1710 may be daisy chained with a length of extensible cord similar to extensible cord 1712 coupled each of the socket assemblies to a neighboring socket assembly. In a related embodiment, a first socket assembly is coupled to a power cord 1730 and has a receptacle for coupling a section of extensible cord like extensible cord 1712 to which a second socket assembly is coupled. In this embodiment, each of the socket assemblies other than the first socket assembly may include two receptacles to receive a section of extensible cord like extensible cord 1712 from neighboring socket assemblies. In this way, two or more socket assemblies may be daisy chained such that electrical current is passed between each socket assembly and its neighbor.

FIG. 17 depicts the adjustable germicidal system 1700 installed at the wall of duct 1760. When an air carrying duct or other air handling apparatus is in a stepped configuration or has multiple angles, such as the stepped duct 1762 shown in FIGS. 18 and 19, the adjustable germicidal system 1700 may be attached to a duct in a multitude of ways so as to maximize the position of the lamps 1720 to achieve the desired germicidal effect. In example configurations, adjustable germicidal system 1700 is attached to conform to the square or angled comers of the duct. In this configuration, the lamps 1720 may be set at desired locations by extending extensible coiled cord 1712 of the germicidal system 1700. For example, the socket assemblies 1710 may be attached to a side 1764 of an air carrying duct 1762, as shown in FIG. 18. In another example, the socket assemblies 1710 may be attached to panels 1766 of an air carrying duct 1762, as shown in FIG. 19. In yet another example, not shown, one of the socket assemblies 1710 may be attached to a panel 1766 while the other socket assembly is attached to side 1764.

The extensible coiled cord 1712 may be manufactured from any resilient, strong, durable material that is flexible and remains functional despite temperature changes within a normal operating temperature range, such as, for example, but not limited to, plastics, rubbers, metals, resins, other materials and combinations thereof.

The germicidal system 1700 may receive electrical power from a single power cord 1730. The extensible coiled cord 1712 has incorporated therein an internal power channel, line or other power carrying medium. In this way electrical power may be passed from one socket assembly 1710 to the other socket assembly 1710, such that both socket assemblies 1710 receive electrical power from a single power source. The same electrical transmission technique may be used in embodiments having three or more socket assemblies.

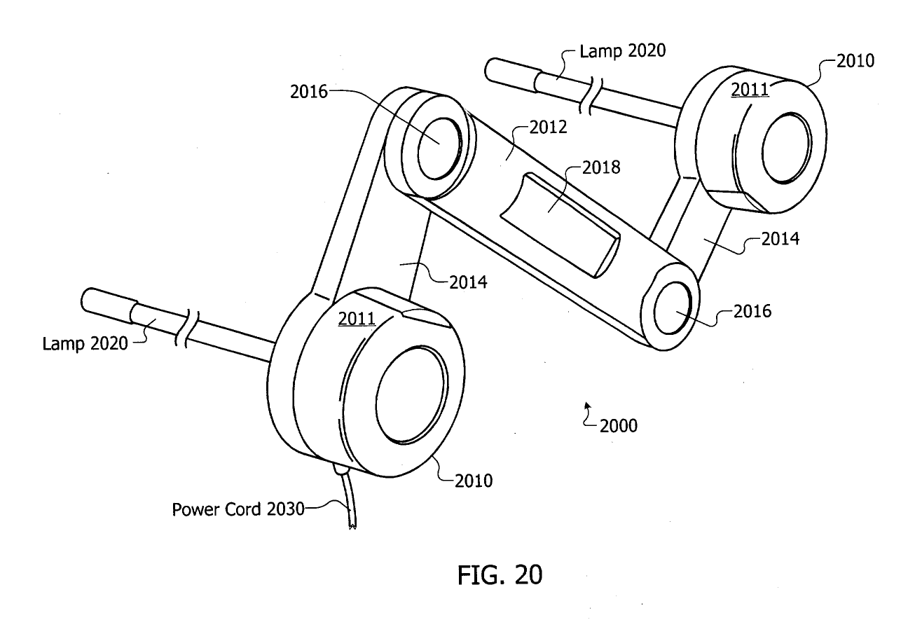

FIG. 20 is a top perspective view of an adjustable germicidal system 2000 having a cross arm 2012 attached with hinges 2016 to socket assemblies 2010. A lamp 2020 is attached to each of socket assemblies 2020. Each of socket assemblies includes arm 2014 by which the socket assembly 2010 is movably coupled to cross arm 2012 via hinge 2016. The hinged connection between cross arm 2012 and the arms 2014 of socket assemblies 2010 allows the lamps 2020 to be adjusted in relation to one another by moving the socket assemblies 2010 in two axes in relation to one another. Arms 2014 of socket assemblies 2010 extend a sufficient distance from the circular portion 2011 of socket assembly 2010 to allow the socket assemblies 2010 to be adjusted in two dimensions in relation to one another.

As to cross arm 2012, although only one cross arm 2012 is depicted in FIG. 20, in other embodiments, two or more cross arms 2012 may be coupled to one another via hinges between two socket assemblies 2010. In addition, cross arm 2012 is shown within area 2018. In addition, the length of cross arm 2012 may be any desired length that allows for placing the socket assemblies 2010 in position for attaching the germicidal system 2000 to the walls of a variety of air handling apparatus.

The lamps 2020 may receive electrical power via a single power cord 2030.

In a related embodiment, the adjustable germicidal system 2000 may be constructed such that hinges 2016 are replaced with ball joints or other spatially adjustable attachment mechanism. Ball joints or other spatially adjustable attachment mechanism allow the socket assemblies to be adjusted in three dimensions with relation to one another. This allows lamps 2020 to be moved in three dimensions along three axes with relation to one another.

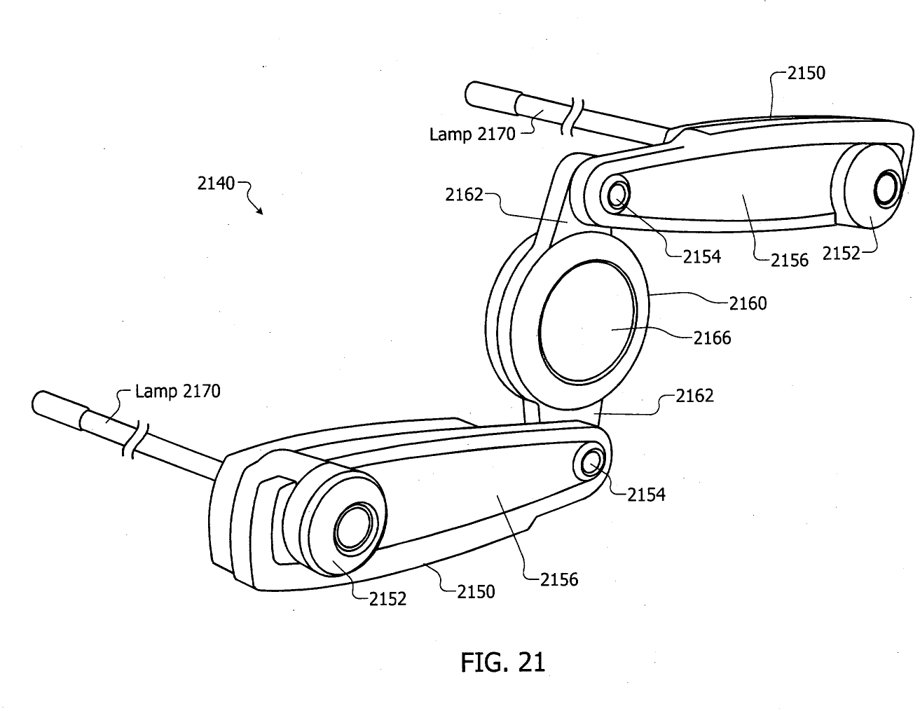

FIG. 21 is atop perspective view of an adjustable germicidal system 2140 having a circular cross panel 2160 attached with hinges 2154 to elongated socket assemblies 2150. In one embodiment, each of the socket assemblies 2150 include a lamp holder 2152. A lamp 2170 is attached to each of lamp holders 2152. In one embodiment in which the lamps 2170 are not integrated lamps, the lamp holder 2152 may include a ballast unit.

Each of socket assemblies 2150 includes elongated arm 2156 by which the socket assembly 2150 is coupled to circular cross panel 2160. In one embodiment, the socket assembly 2150 is movably coupled via hinge 2154 to an arm 2162 of circular cross panel 2160. The hinged connections 2154 between circular cross panel 2160 and the elongated arms 2156 of socket assemblies 2150 allow the lamps 2170 to be adjusted in two dimensions. That is, the socket assemblies 2150 move in two axes in relation to one another. The elongated arms 2156 of socket assemblies 2150 extend a sufficient distance from the ballast unit 2152 to allow the socket assemblies 2150 to be adjusted in two dimensions in relation to one another.

In one embodiment, the circular cross panel 2160 includes arms 2162 that are fixed in relation to circular portion 2166. In another embodiment, the hinges 2154 are replaced with fixed couplings, and the arms 2162 are movably coupled to circular portion 2166. In this way, cross panel 2160 rotates at circular portion 2166 which acts as a hinge such that the arms 2162 rotate in a plane about circular portion 2166.

As to circular cross panel 2160, although only one circular cross panel 2160 is depicted in FIG. 21, in other embodiments, two or more cross panels may be coupled to one another via hinges. In addition, the length of arms 2162 may be any desired length that allows for placing the lamps 2170 of socket assemblies 2150 in position for attaching the germicidal system 2140 to a variety of air handling apparatus.

The adjustable germicidal system 1240 is attached to a wall of an air handling apparatus. In one embodiment, only circular cross panel 2160 is affixed to a wall of an air handling apparatus. In another embodiment, each of the socket assemblies 2150 is affixed to a wall of an air handling apparatus. In yet another embodiment, circular cross panel 2160 and each of the socket assemblies 2150 are affixed to a wall of an air handling apparatus.

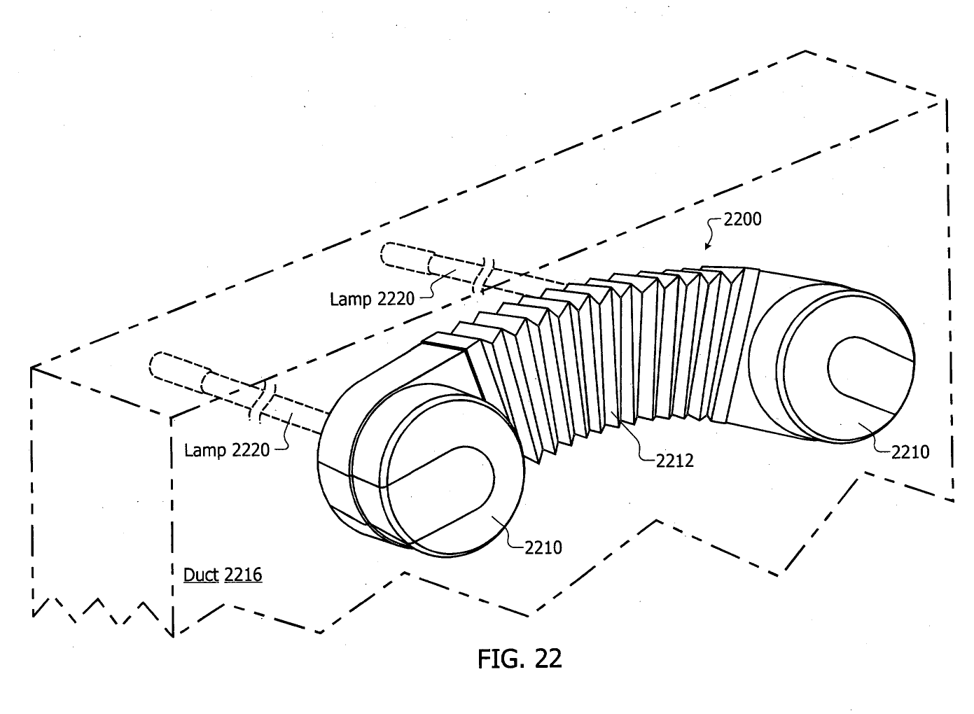

FIG. 22 is a top perspective view of an adjustable germicidal system 2200 having an accordion portion 2212 shown in a partially extended and curved position attached to a wall 2216 of an air handling apparatus. In this embodiment, socket assemblies 2210 are coupled to one another via an extendable accordion portion 2212. The extendable accordion portion 2212 allows the lamps 2220 to be adjusted in two or three dimensions in relation to one another. In this way, the lamps 2220 may be positioned a desired distance apart while taking into consideration placement limitations resulting from the air handling apparatus.

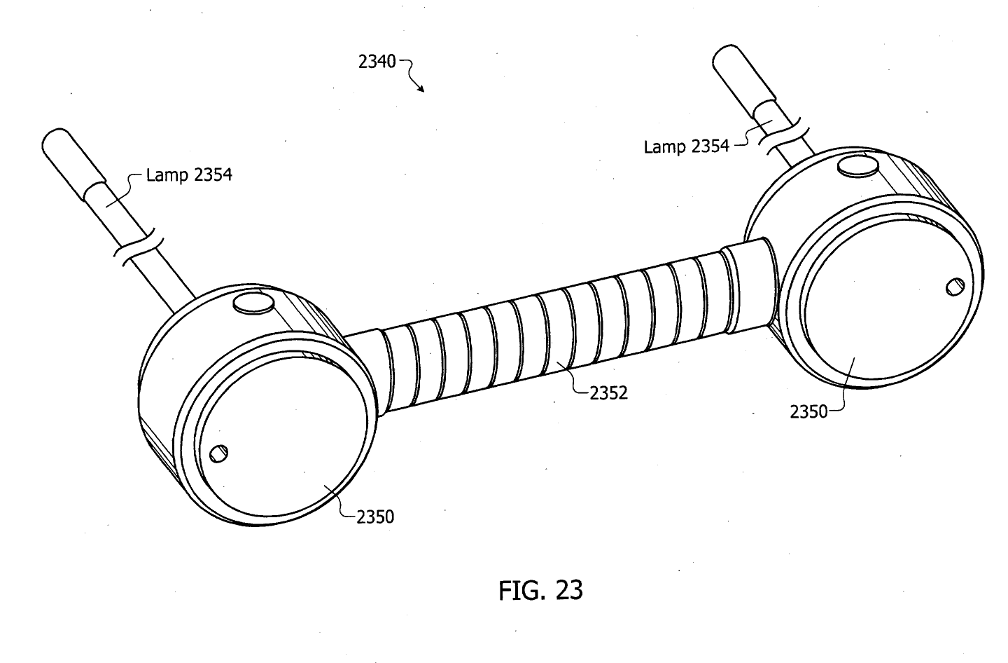

FIG. 23 is a top perspective view of an adjustable germicidal system 2340 having a flexible tube 2352 connecting the socket assemblies 2350. In this embodiment, socket assemblies 2350 are coupled to one another via flexible tube 2352 which allows the lamps 2354 to be adjusted in two or three dimensions in relation to one another. In this way, the lamps 2320 may be positioned a desired distance apart without tools.

Although exemplary embodiments of the invention have been shown and described, it will be apparent to those having ordinary skill in the art that a number of changes, modifications, or alterations to the invention as described herein may be made, none of which depart from the spirit of the invention. All such changes, modifications and alterations should therefore be seen as within the scope of the invention.

It is claimed:

1. An adjustable germicidal system comprising:
a first socket assembly adjustably coupled to a second socket assembly
a first germicidal lamp coupled to the first socket assembly
a second germicidal lamp coupled to the second socket assembly
the relative position of the first germicidal lamp and the second germicidal lamp may be adjusted by spatially arranging the first and second socket assemblies prior to attachment of the first and second socket assemblies to a first side of a wall, the first germicidal lamp and the second germicidal lamp disposed on a second side of the wall
a single electrical power cord coupled to the first socket assembly.

2. The adjustable germicidal system of claim 1 wherein the first socket assembly and the second socket assembly are attached to the first side of the wall and pass through the second side of the wall.

3. The adjustable germicidal system of claim 1 wherein the first socket assembly and the second socket assembly are attached to the first side of the wall, and the first germicidal lamp and the second germicidal lamp pass from the first side of the wall to the second side of the wall.

4. The adjustable germicidal system of claim 1 wherein the first socket assembly and the second socket assembly may be moved in a single dimension in relation to one another.

5. The adjustable germicidal system of claim 1 wherein the first socket assembly and the second socket assembly may be moved in two dimensions in relation to one another. The adjustable germicidal system of claim 1 wherein the first socket assembly and the second socket assembly may be moved in three dimensions in relation to one another.

6. The adjustable germicidal system of claim 1 wherein the second socket assembly receives electrical power through the first socket assembly.

7. The adjustable germicidal system of claim 1 wherein the first socket assembly is coupled to the second socket assembly via an extensible cord.

8. The adjustable germicidal system of claim 1 wherein the first socket assembly is coupled to the second socket assembly via a flexible tube.

9. The adjustable germicidal system of claim 1 wherein the first socket assembly is coupled to the second socket assembly via a cross arm having hinged connections to the first socket assembly and to the second socket assembly.

10. The adjustable germicidal system of claim 1 wherein the first socket assembly and the second socket assembly have two slotted edges and are included in a housing having two receiving slots which correspond with the slotted edges, the slotted edges of the socket assemblies and the corresponding receiving slots allow the first socket assembly and the second socket assembly to be adjusted in a single axis with regard to one another within the housing.

11. The adjustable germicidal system of claim 1 wherein the first socket assembly is coupled to the second socket assembly via at least two hinged panels to allow for adjustment of the first germicidal lamp and the second germicidal lamp in two axes.

12. The adjustable germicidal system of claim 1 wherein the first socket assembly is coupled to the second socket assembly via at least two telescoping portions.

13. The adjustable germicidal system of claim 1 wherein the first socket assembly is coupled to the second socket assembly via an accordion portion.

14. The adjustable germicidal system of claim 1 wherein the first and second germicidal lamps emit ultraviolet radiation substantially in the range of 250-260 nm and generate an insignificant quantity or less of ozone.

15. The adjustable germicidal system of claim 1 wherein the first and second germicidal lamps are single ended.

16. An air treatment unit having the adjustable germicidal system of claim 1 coupled thereto.

17. An air carrying duct having the adjustable germicidal system of claim 1 coupled thereto.

18. The adjustable germicidal system of claim 1 wherein the first socket assembly is coupled to the second socket assembly via at least two telescoping portions.

19. The adjustable germicidal system of claim 1 wherein the first socket assembly is coupled to the second socket assembly via an accordion portion.

20. An adjustable germicidal system comprising:
at least two socket assemblies adjustably coupled in series to one another, the at least two socket assemblies to be coupled to a first side of a wall
at least two germicidal lamps disposed on a second side of a wall and coupled to the at least two socket assemblies, the number of germicidal lamps corresponding to the number of socket assemblies, the germicidal lamps spatially adjustable with respect to the other germicidal lamps
a single electrical power cord coupled to one of the socket assemblies.

21. The adjustable germicidal system of claim 20 wherein the socket assemblies are attached to the first side of the wall and pass through the second side of the wall.

22. The adjustable germicidal system of claim 20 wherein the socket assemblies are attached to the first side of the wall, and the germicidal lamps pass from the first side of the wall to the second side of the wall.

23. The adjustable germicidal system of claim 20 wherein the germicidal lamps may be moved in a single axis in relation to one another.

24. The adjustable germicidal system of claim 20 wherein the germicidal lamps may be moved in two axes in relation to one another.

25. The adjustable germicidal system of claim 20 wherein the germicidal lamps may be moved in three axes in relation to one another.

26. The adjustable germicidal system of claim 20 wherein a first socket assembly of the at least two socket assemblies is coupled to an electrical power source and each successive socket assembly receives the electrical power in series from its neighbor.

27. The adjustable germicidal system of claim 20 wherein the germicidal lamps emit ultraviolet radiation substantially in the range of 250-260 nm and generate an insignificant quantity or less of ozone.

28. The adjustable germicidal system of claim 20 wherein the germicidal lamps are single ended.

29. An air treatment unit having the adjustable germicidal system of claim 20 coupled thereto.

30. An air carrying duct having the adjustable germicidal system of claim 20 coupled thereto.

31. A method of configuring germicidal systems comprising:
selecting a wall of an air handling apparatus
creating at least two holes in the wall of the air handling apparatus
attaching on the wall a germicidal system comprising at least two germicidal lamps and corresponding socket assemblies, the socket assemblies adjustably coupled to each other in series, the attaching including positioning the socket assemblies on a first side of the wall such that the at least two germicidal lamps are disposed on a second side of the wall
connecting one of the socket assemblies to a power source to energize all of the germicidal lamps to emit ultraviolet light
irradiating an internal structure of the air handling apparatus and air within the air handling apparatus.

32. The method of claim 31 wherein the positioning comprises
moving the socket assemblies closer together or farther apart in a single plane so that the germicidal lamps may be moved relative to one another.

33. The method of claim 31 wherein the positioning comprises
placing the socket assemblies in at least two different planes so that the germicidal lamps are in two different planes.

34. The method of claim 31 wherein the positioning comprises
moving the socket assemblies in up to three axes to place the germicidal lamps in an alignment within the air handling apparatus to maximize the irradiating.

35. A method of configuring germicidal systems comprising:
selecting an air handling apparatus having multiple walls
create at least two holes through at least two of the multiple walls
attaching a germicidal system to the air handling apparatus, the germicidal system comprising at least two germicidal lamps and corresponding socket assemblies, the socket assemblies adjustably coupled to each other in series, the attaching including positioning the socket assemblies on a first side of at least two of the multiple walls such that the lamps are disposed on a second side of at least two of the multiple walls
connecting one of the socket assemblies to a power source to energize all of the germicidal lamps to emit ultraviolet light
irradiating the internal structure of the air handling apparatus and air within the air handling apparatus.

36. The method of claim 35 wherein the positioning comprises
moving the socket assemblies closer together or farther apart in a single plane so that the germicidal lamps may be moved relative to one another.

37. The method of claim 35 wherein the positioning comprises
placing the socket assemblies in at least two different planes so that the germicidal lamps are in two different planes.

38. The method of claim 35 wherein the positioning comprises
moving the socket assemblies in up to three axes to place the germicidal lamps in a desired alignment within the air handling apparatus.

39. An adjustable germicidal system comprising:
at least two means for emitting ultraviolet light to eradicate germs in a means for handling air
at least two means for receiving the at least two means for emitting ultraviolet light, the at least two means for receiving spatially adjustably coupled in series to one another, the at least two means for receiving to be coupled to a first side of a wall of the means for handling air such that the at least two means for emitting ultraviolet light are disposed on a second side of the wall of the means for handling air to emit ultraviolet light within the means for handling air a single means for receiving electrical power coupled to one of the means for receiving.

40. An adjustable germicidal system comprising:
a first socket assembly adjustably coupled to a second socket assembly, the first socket assembly configured to receive a first germicidal lamp and the second socket assembly configured to receive a second germicidal lamp
the relative position of the first socket assembly and the second socket assembly may be adjusted by spatially arranging the first and second socket assemblies prior to attachment of the first and second socket assemblies to a first side of a wall of an air handling apparatus immediately prior to installation of the germicidal system on the wall
a single electrical power cord coupled to the first socket assembly.

41. The adjustable germicidal system of claim 40 wherein the first socket assembly and the second socket assembly may be moved in a single dimension in relation to one another.

42. The adjustable germicidal system of claim 40 wherein the first socket assembly and the second socket assembly may be moved in two dimensions in relation to one another.

43. The adjustable germicidal system of claim 40 wherein the first socket assembly and the second socket assembly may be moved in three dimensions in relation to one another.

44. The adjustable germicidal system of claim 40 wherein the second socket assembly receives electrical power through the first socket assembly.

45. The adjustable germicidal system of claim 40 wherein the first socket assembly is coupled to the second socket assembly via an extensible cord.

46. The adjustable germicidal system of claim 40 wherein the first socket assembly is coupled to the second socket assembly via a flexible tube.

47. The adjustable germicidal system of claim 40 wherein the first socket assembly is coupled to the second socket assembly via a cross arm having hinged connections to the first socket assembly and to the second socket assembly.
